(19) United States
(12) Patent Application Publication
(10) Pub. No.: US 2026/0060796 A1
(43) Pub. Date: Mar. 5, 2026

(54) AN ADJUSTABLE MULTI-LOOP FIXATION DEVICE

(71) Applicant: BIORAD MEDISYS PRIVATE LIMITED, Pune (IN)

(72) Inventors: Debasish PRADHAN, Pune (IN); Pranay BHAVSAR, Pune (IN)

(21) Appl. No.: 19/108,180

(22) PCT Filed: Sep. 1, 2023
(86) PCT No.: PCT/IN2023/050827

§ 371 Date: Mar. 3, 2025

(30) Foreign Application Priority Data
Sep. 2, 2022 (IN) 202221050188

(51) Int. Cl. A61F 2/08

(52) U.S. Cl. CPC A61F 2/0811; A61F 2002/0852; A61F 2002/0882

(57) ABSTRACT

The present subject disclosed a device for tissue repairs and reconstruction of anterior cruciate ligament (ACL) and posterior cruciate ligament (PCL). Further the device comprises a fixation plate having an upper side and a lower side, the upper side comprising a plurality of circular through holes, an elliptical groove with apertures connected by a bridge, and a hole with a step extending from the upper side to the lower side. In the embodiment, the device comprises further a strand, the strand comprises a knot. The knot divides the strand into a tension end and a lead end.

### FIELD OF THE PRESENT SUBJECT MATTER

The present subject matter relates generally to the field of medical devices. More particularly, the present subject matter relates to a device for tissue repairs and reconstruction techniques, and associated fixation and reconstruction such as surgical reconstruction of Anterior Cruciate Ligament (ACL) and Posterior Cruciate Ligament (PCL).

### BACKGROUND

A ligament is a piece of fibrous tissue which connects one bone to another. Ligaments are frequently damaged (e.g., detached or torn or ruptured, etc.) because of injury and/or accident. A damaged ligament can impede proper motion of a joint and cause significant pain. One of the most common types of such injuries is tearing of the Anterior Cruciate Ligament (ACL). The ACL connects the femur to the tibia at the centre of the knee joint. A damaged ACL can cause instability of the knee joint and cause substantial pain and arthritis. Surgical techniques have been developed to reconstruct such soft tissues and to re-attach them to the relevant bone.

Reconstructive surgeries, particularly anterior cruciate ligament (ACL) reconstruction, are well-known in the art. It is generally accepted in the field of orthopaedic surgery that the anterior cruciate ligament does not heal itself after injury. Initial attempts at repair of this ligament resulted in nearly uniform failure of the ligament to stabilize the knee joint. Over the course of decades, practitioners have turned to methods of ligament reconstruction in attempts to restore knee stability and normal knee kinematics. Cortical fixation may be preferred by surgeons who primarily use soft tissue grafts. Sutures and various fixation devices (e.g., buttons, anchors, etc.) are commonly used in the field of orthopaedic surgery for performing joint stabilizations, tissue repairs, tissue reconstructions, fracture repairs, and other similar surgical methods. These types of surgical methods often involve fixating damaged tissue or realigning bones to restore the functionality of a joint.

In ACL procedures the elongated member, often called a button, is adapted on the anterolateral femoral cortex which is attached to the graft by means of sutures or tapes through a tunnel in the femur. A particularly important issue in the grafting operation is that the graft is fixed in the bone tunnel at the required tension. Inappropriate tensioning of the graft will affect the success of the operation. In practice, tensioning devices may be utilised but having achieved the required tension, it is then necessary for the surgeon to tie off the suture and the tying of knots introduces errors into the process and may result in some subsequent stretch in the suture causing an inappropriate tension in the graft. Sometimes, adjustable loops lead to extension of loop under graft tension due to higher tension at lock arrangement resulting in the gradual sliding of end suture (tensioning/adjustment suture) under sling lock. Adjustable suture loops at times require pulling with high forces on the loops to reduce them, which can sometimes injure the surgeon or damage the surgeon's gloves. Some attempted solutions have included wrapping sutures multiple times around an instrument such as a forceps or using reinforced gloves, but this has not sufficiently addressed the needs of the industry.

Accordingly, convention devices fail to provide for simplified attachment of tendons or ligaments to fixation devices using suturing techniques to achieve the required forces and have maximum fixation strength.

### SUMMARY

Embodiment of the present disclosure presents technological improvements as solutions to one or more of the above-mentioned technical problems recognized by the inventor(s) in conventional systems.

In one embodiment, and device for tissue repairs and reconstruction of anterior cruciate ligament (ACL) and posterior cruciate ligament (PCL) is disclosed. Further the device comprises a fixation plate having an upper side and a lower side, the upper side comprising a plurality of circular through holes, an elliptical groove with twin apertures connected by a bridge, and a hole with a step extending from the upper side to the lower side. In the embodiment, the device comprises further a strand, the strand comprises a knot. The knot divides the strand into a tension end and a lead end. The tension end is passed through the hole in the fixation plate from the lower side to the upper side and through one of the twin apertures from the upper side to the lower side, forming a first adjustable fixation loop. The tension end of the strand further passed through the aperture from the lower side to the upper side, forming a first small loop. The tension end of the strand passed through the hole from the lower side to the upper side, forming a second adjustable fixation loop with the tension end and lead end extending away from the upper side. The tension end of the strand further passed through the aperture from the upper side to the lower side, forming a second small loop. The tension end of the strand passed through the hole from the lower side to the upper side, forming a third adjustable fixation loop. The tension end of the strand is passed through the first small loop to lock the adjustable loops, and the lead end of the strand is pulled through the hole and a guide way at the lower side, then pulled out through a second through hole at the upper side.

In one embodiment, a method for attaching a graft, comprising the steps of: providing a device, the device comprising a fixation plate having an upper side and a lower side, the upper side comprising a plurality of circular through holes, an elliptical groove with twin apertures connected by a bridge, and a hole with a step extending from the upper side to the lower side, and a flexible single strand having a knot dividing it into a tension end and a lead end; passing the tension end of the strand through the hole in the fixation plate from the lower side to the upper side, then through one of the twin apertures from the upper side to the lower side, forming a first adjustable fixation loop; passing the tension end of the strand through the aperture from the lower side to the upper side, forming a first small loop; passing the tension end of the strand through the hole from the lower side to the upper side, forming a second adjustable fixation loop with the tension end and lead end extending away from the upper side; passing the tension end of the strand through the aperture from the upper side to the lower side, forming a second small loop; passing the tension end of the strand through the hole from the lower side to the upper side, forming a third adjustable fixation loop; passing the tension end of the strand through the first small loop to lock the adjustable loops, and pulling the lead end of the strand through the hole and a guide way at the lower side, then pulling the lead end out through a second through hole at the upper side; attaching a graft to the first, second, and third adjustable fixation loops of the fixation device; and applying tension to the tension end of the strand, shortening the adjustable loops, and transferring load onto the graft, thereby achieving graft fixation, and minimizing slip/creep of the strand within the loops.

It will be appreciated that features of the present disclosure are susceptible to being combined in various combinations without departing from the scope of the present disclosure as defined by the detailed description and drawings below.

### BRIEF DESCRIPTION OF THE FIGURES

The summary above, as well as the following detailed description of illustrative embodiment, is better understood when read in conjunction with the appended drawings. For illustrating the present disclosure, example constructions of the disclosure are shown in the drawings. However, the present disclosure is not limited to specific methods and instrumentalities disclosed herein. Moreover, those in art will understand that the drawings are not too scale. Wherever possible, like elements have been indicated by identical numbers.

Embodiment of the present disclosure will now be described, by way of example only, with reference to the following diagrams wherein:

FIG. 1. Illustrates a perspective view of the fixation device in accordance with an exemplary embodiment of the present disclosure;

FIG. 2(a)-2(d) illustrates a perspective view of sides of fixation plate and a single braided medical strand element combined to form a fixation device in accordance with an exemplary embodiment of the present disclosure;

FIG. 3(a)-3(j) illustrates the steps of building the embodiment of fixation device with an exemplary embodiment of the present disclosure;

FIG. 4(a)-4(c) illustrates a graft connected to the adjustable fixation loops of the fixation device with an exemplary embodiment of the present disclosure.

In the above accompanying drawings, a number relates to an item identified by a line linking the number to the item. When a number is accompanied by an associated arrow, the number is used to identify a general item at which the arrow is pointing.

Further the figures depict various embodiments of the present subject matter for purposes of illustration only. One skilled in the art will readily recognize from the following discussion that alternative embodiment of the structures and methods illustrated herein may be employed without departing from the principles of the present subject matter described herein.

### DETAILED DESCRIPTION OF EMBODIMENTS

The following detailed description illustrates embodiment of the present disclosure and manners by which they can be implemented. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

The person skilled in the art will recognize many variations, alternatives, and modifications of the embodiment of the present disclosure. It should be understood that this present subject matter is not limited to the particular methodology, protocols, and the like, described herein and as such may vary. The terminology used herein is for the purpose of describing embodiment only and is not intended to limit the scope of the present subject matter, which is defined solely by the claims.

As discussed above, Orthopedic medicine, dedicated to diagnosing, treating, and preventing disorders related to the bones, joints, and ligaments, holds paramount importance in ensuring that humans can move with ease and without pain. One of the focal points in this realm of medicine is the treatment and management of ligament injuries. Ligaments, the flexible but tough connective tissues that link bones together, are pivotal in ensuring joint stability and seamless movement.

However, it's unfortunate that these critical structures are susceptible to injuries. When we think of ligament damage, one of the most common and severe injuries that comes to mind is the tearing of the Anterior Cruciate Ligament (ACL) in the knee. Such injuries not only destabilize the knee but can also pave the way for subsequent problems, such as chronic pain and arthritis. With the knee being central to most of our movements, any issue with its functionality can severely impede daily activities.

Conventionally, attempts to heal torn ACLs have been fraught with challenges. For starters, the ACL is a stubborn ligament. Unlike some other tissues in the body, it doesn't have the intrinsic capability to self-repair or regenerate. When left untreated, a torn ACL would leave the knee unstable indefinitely. Therefore, orthopedic surgeons have always been in pursuit of innovative techniques to effectively mend this ligament and restore the knee's functionality.

In a conventional method, simply stitching the torn ligament back together was used. However, the results were far from ideal. The repaired ligament often failed to provide the required stability, making the surgery less effective. Realizing the shortcomings of this approach, medical professionals ventured into other conventional techniques and methodologies. Their endeavors led to the advent of reconstructive surgeries, which essentially involve using grafts (either from the patient's body or a donor) to replace the damaged ligament.

To anchor these grafts securely in place, surgeons employ an array of conventional tools and materials, from surgical buttons to intricate stitches. The objective is clear: to ensure that the newly installed graft mimics the strength and functionality of a natural, healthy ACL. Here conventional technologies fail, especially in getting the tension right.

Ensuring that the graft has the appropriate tension is imperative for optimal knee stability. However, it's easier said than done. Many existing conventional techniques have various challenges. Applying the right amount of tension can be physically demanding for the surgeon. The sheer force required can not only be exhausting but can also pose risks, such as damage to surgical gloves, potentially compromising the sterile environment.

Some surgeons have experimented with wrapping the sutures around instruments or wearing reinforced gloves to counteract these challenges. Yet, these solutions have been more like band-aids, providing temporary relief without addressing the root cause.

Thus, one object of the present subject matter is devising a device that ensures the graft remains as taut as a natural ligament without overburdening the surgeon or compromising the sterility of the operation. Achieving this could revolutionize ACL surgeries, making them more effective and ensuring better outcomes for countless patients worldwide. Further present subject matter is therefore directed towards providing an improved method and device, which is relatively simple in design and structure, and is highly effective for its intended purpose.

One other object of the present subject matter is providing a device and method to anchor ligaments or tendons, like the ACL, that can withstand the necessary tension without compromising the graft's integrity or the surgeon's convenience. Thus, the present subject matter would not only enhance the success rate of ACL surgeries but would also significantly improve post-surgical outcomes for patients.

An object of the present subject matter is to provide an improved and novel construction of a fixation device which is designed to overcome the disadvantages of the known existing devices for ACL reconstruction thereby minimizing slip/creep of the braided medical strand within the loop.

Another object of the present subject matter is to provide an improved method for reconstructing a ligament by using an adjustable loop which simplifies the procedure and maximize the bone to soft tissue interface.

Yet another object of the present subject matter is to provide a fixation device and a method to attach to an adjustable fixation loop of braided medical strand without compromising the graft or requiring additional material to complete the repair.

The present subject matter provides techniques and reconstruction systems for fixation of bone to bone, or soft tissue to bone. The reconstruction system of the present subject matter discloses a fixation device comprising a fixation plate and a single length flexible and adjustable braided medical strand and a method to assemble them and connect to tissue. In an aspect of the present disclosure, to make a fixation device, The braided medical strand and fixation plate is assembled by following a tortuous path through the various apertures of the fixation plate. In order to achieve this, the tension side of braided medical strand is passed multiple times through the twin apertures connected via bridge extending between them inside the elliptical groove, and a hole with a step which is connected to the first aperture via a channel eventually pulling out the lead end of the braided medical strand from a through hole placed at the corner of the fixation plate via a guide way at the bottom side of the fixation plate. The braided medical strand and fixation device is assembled outside the body of the patient. The graft is attached to the three adjustable fixation loops of the fixation device. The fixation device is pulled out through the bone tunnel during surgery by pulling the lead end of braided medical strand whereas, the tension end of the braided medical strand is pulled to adjust the tension of the graft into the bone tunnel by shortening the three adjustable fixation loops thereby achieving the maximum fixation and minimizing slip/creep of the braided medical strand within the loop. The present subject matter particularly works on the principle which resembles like a pulley for shortening of the adjustable fixation loops and transferring the load on the graft.

In one embodiment, and device for tissue repairs and reconstruction of anterior cruciate ligament (ACL) and posterior cruciate ligament (PCL) is disclosed. In one advantage the device is an adjustable multi-loop fixation device. Further the device comprises a fixation plate having an upper side and a lower side, the upper side comprising a plurality of circular through holes, an elliptical groove with twin apertures connected by a bridge, and a hole with a step extending from the upper side to the lower side. Further, in one example the fixation plate is made from a material selected from the group consisting of plastic, titanium alloy, and steel.

In one embodiment, the fixation plate has curved edges to facilitate smooth pulling of the fixation device through a bone tunnel during surgery. Further, the circular through holes is disposed at corners of the fixation plate and the bridge connecting the twin apertures forms a twisted path for the strand.

In the embodiment, the device comprises further a strand. In one example, the strand is flexible, single braided, medicated and shaped in a circular cross-section. In one more example, the strand is made of Ultra High Molecular Weight polyethylene (UHMWPE) or a biocompatible material. The strand comprises a knot. The knot divides the strand into a tension end and a lead end. The step within the hole is configured to arrest the knot in the strand, preventing its slippage during tension.

The tension end is passed through the hole in the fixation plate from the lower side to the upper side and through one of the twin apertures from the upper side to the lower side, forming a first adjustable fixation loop. The tension end of the strand further passed through the aperture from the lower side to the upper side, forming a first small loop. The tension end of the strand passed through the hole from the lower side to the upper side, forming a second adjustable fixation loop with the tension end and lead end extending away from the upper side. The tension end of the strand further passed through the aperture from the upper side to the lower side, forming a second small loop. The tension end of the strand passed through the hole from the lower side to the upper side, forming a third adjustable fixation loop. The tension end of the strand is passed through the first small loop to lock the adjustable loops, and the lead end of the strand is pulled through the hole and a guide way at the lower side, then pulled out through a second through hole at the upper side.

In the embodiment the device comprises a graft connected to the first, second, and third adjustable fixation loops, wherein tension applied to the tension end of the strand shortens the adjustable loops, achieving graft fixation and minimizing slip/creep of the strand within the loops.

In the embodiment the tension end of the strand is slidably adjustable within the twin apertures to achieve desired tension in the graft.

In the embodiment the tension end of the strand is pulled to shorten the adjustable loops, distributing the load on the graft, and achieving desired fixation.

In one other embodiment, a method for attaching a graft, comprising the steps of: providing a device, the device comprising a fixation plate having an upper side and a lower side, the upper side comprising a plurality of circular through holes, an elliptical groove with twin apertures connected by a bridge, and a hole with a step extending from the upper side to the lower side, and a flexible single strand having a knot dividing it into a tension end and a lead end; passing the tension end of the strand through the hole in the fixation plate from the lower side to the upper side, then through one of the twin apertures from the upper side to the lower side, forming a first adjustable fixation loop; passing the tension end of the strand through the aperture from the lower side to the upper side, forming a first small loop; passing the tension end of the strand through the hole from the lower side to the upper side, forming a second adjustable fixation loop with the tension end and lead end extending away from the upper side; passing the tension end of the strand through the aperture from the upper side to the lower side, forming a second small loop; passing the tension end of the strand through the hole from the lower side to the upper side, forming a third adjustable fixation loop; passing the tension end of the strand through the first small loop to lock the adjustable loops, and pulling the lead end of the strand through the hole and a guide way at the lower side, then pulling the lead end out through a second through hole at the upper side; attaching a graft to the first, second, and third adjustable fixation loops of the fixation device; and applying tension to the tension end of the strand, shortening the adjustable loops, and transferring load onto the graft, thereby achieving graft fixation, and minimizing slip/creep of the strand within the loops.

These and other features and advantages of the present subject matter will become apparent from the following description of the present subject matter that is provided in connection with the accompanying drawings and illustrated embodiments of the present subject matter.

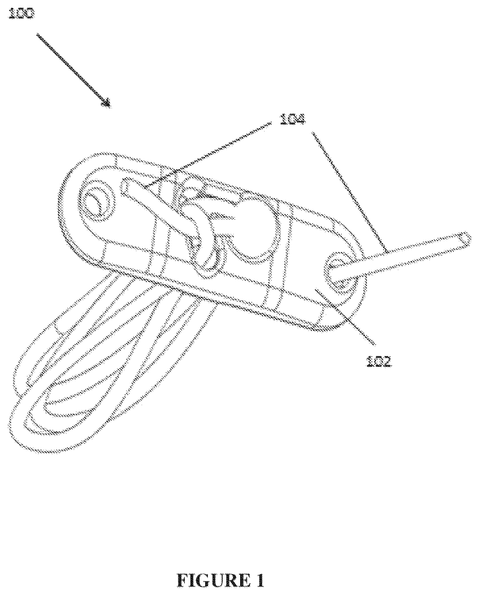

Referring now to the drawings, FIGS. 1, 2, 3, 4 illustrate a fixation device 100 according to present subject matter. It should be noted that FIGS. 1, 2, 3, 4 are merely examples. A person skilled in the art will recognize many variations, alternatives, and modifications of the embodiments of the present disclosure. The fixation device 100 may be any of various types of geometrical shapes like circular, rectangular, trapezoidal and the like according to various embodiments of the present disclosure.

In an aspect of the present disclosure, a fixation device, and a method of building the strand (also referred to as braided medical strand) with the fixation plate is disclosed.

Referring now to the drawings, FIG. 1 illustrates a fixation device 100 comprising a fixation plate 102 and a flexible single braided medical strand 104 together forming the fixation device for ACL reconstruction.

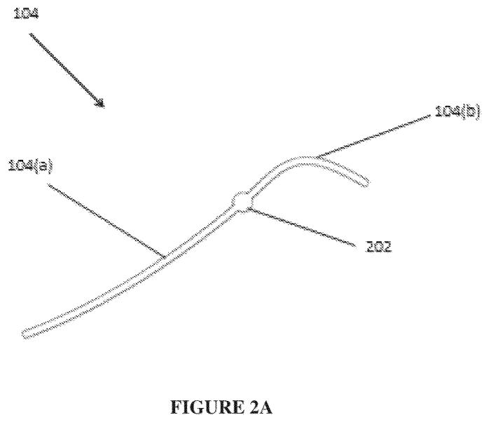

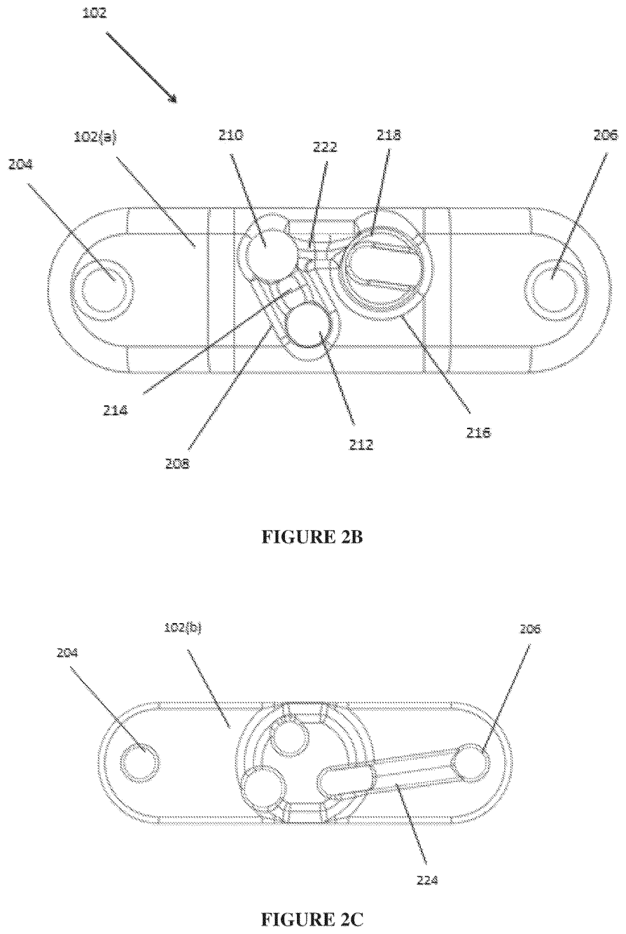

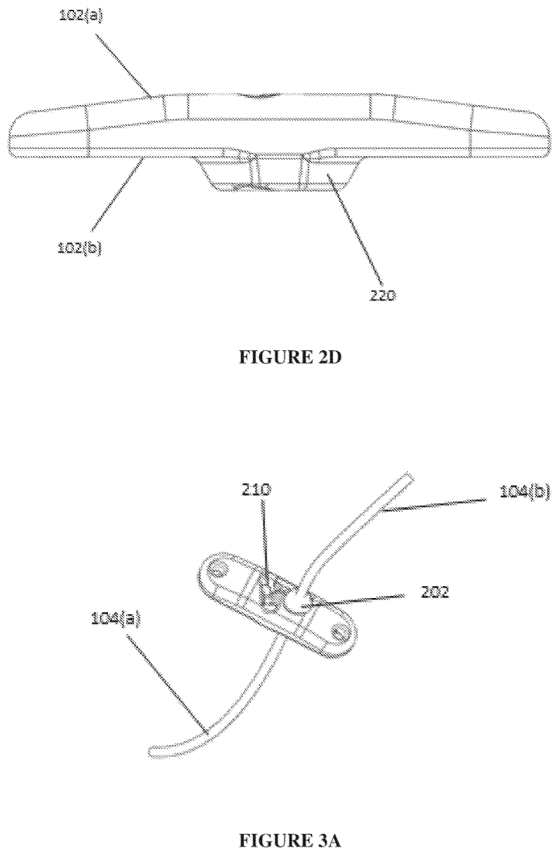

Now referring to FIG. 2(a), the flexible braided medical strand 104 is single in structure and comprises knot 202 which divides the braided medical strand 104 into two parts namely, tension end 104a and lead end 104b. The placement of knot 202 decides the length of the lead end 104b of the braided medical strand. The braided medical strand is made up of Ultra High Molecular Weight polyethylene (UHMWPE) material or bio-compatible material.

Referring to FIG. 2(b), 2(c), and 2(d) the fixation plate 102 has an upper side 102(a) and a lower side 102(b) with curved edges to easily pull the graft into a tunnel without damaging a bone. The upper side 102a of the fixation plate has a plurality of circular through holes 204, 206, an elliptical groove 208 placed at a certain angle with twin apertures 210,212 having a bridge 214 extending between them, a hole 216 comprising of a step 218 extending from the upper side 102a to the lower side 102b of the fixation plate 102 through a dome like structure 220 which acts as a stabilising member. The hole 216 is connected to the aperture 210 through a channel 222.

The through first and second through holes 204 and 206 respectively are placed at the extreme left and right corners on the upper side 102(a) of the fixation plate 102 respectively. The first through hole 204 may be an optional through hole for flipping a braided medical strand which the surgeon may require sometimes to ensure the fixation of device once the fixation device is pulled out of a bone tunnel (explained later). The hole 216 comprise of a step 218 for arresting the knot 202 during the operation ensuring that the knot 202 does not slip down through the hole 216 while applying tensile strength. The second through hole 206 is connected to hole 216 via a guide way 224 at the lower side 102b of the fixation plate 102.

FIG. 2(a), 2(b), 2(c), 2(d) are mere examples. A person skilled in the art will recognize many variations, alternatives, and modifications of the embodiment of the present disclosure. The present subject matter is not limited to the five holes and the number of holes can be more or less than five according to the requirement of the thickness of the fixation plate and the strength to reconstruct the graft to be achieved. The shape of the through holes is not limited to circular and can be of any geometrical shape like triangle, elliptical, rectangle and the like according to the need for the strength during the time of the surgery. The placement of the knot in the braided medical strand is not limited to the embodiment of the present subject matter and can be placed in the middle of the braided medical strand if required. The material used for the braided medical strand is not limited to Ultra High Molecular Weight polyethylene (UHMWPE) material or biocompatible material and can be made of polymer such as polythene or similar material. Furthermore, the braided medical strand can be of different shapes like flat, circular and the like. Moreover, the fixation plate may be made from material like plastic or similar materials and is not limited to the metals having high tensile strength, toughness, corrosion resistance and light in weight like titanium alloy, steels and the like.

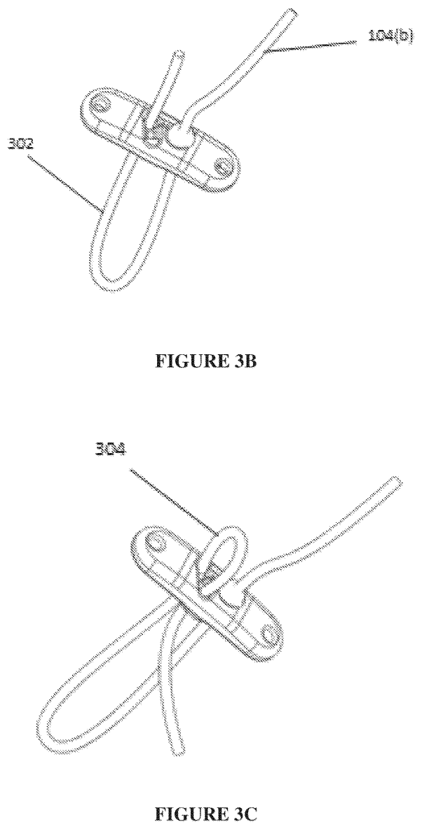

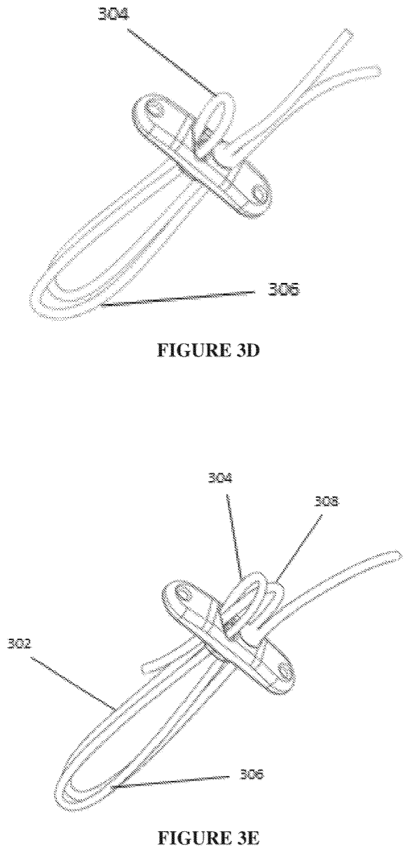

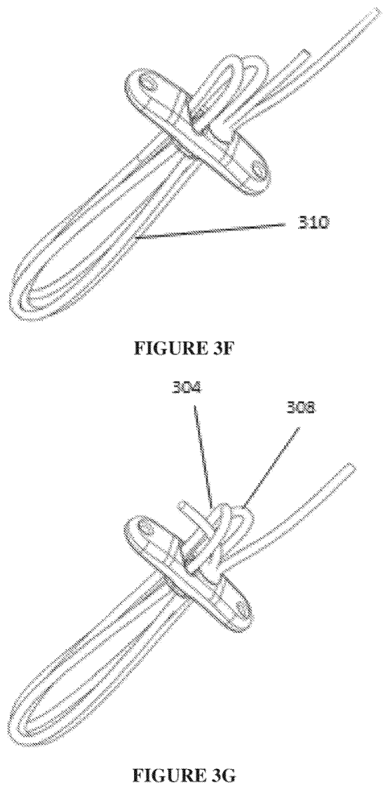

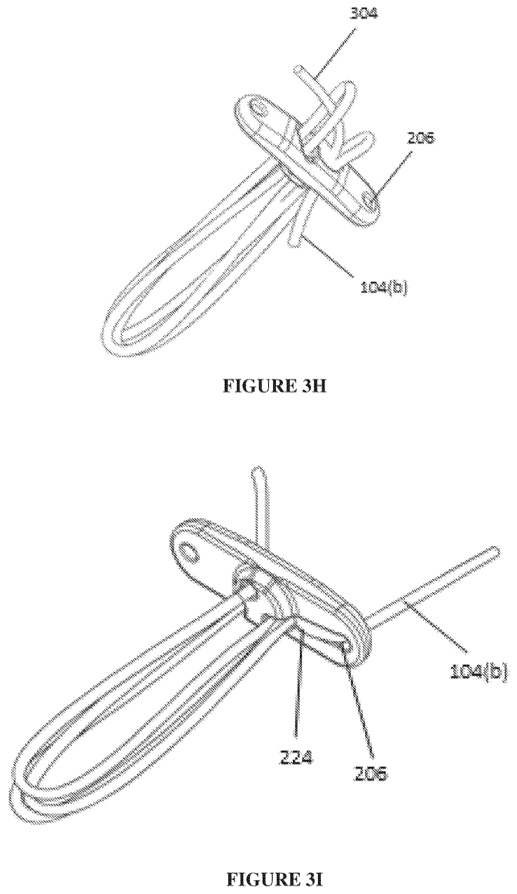

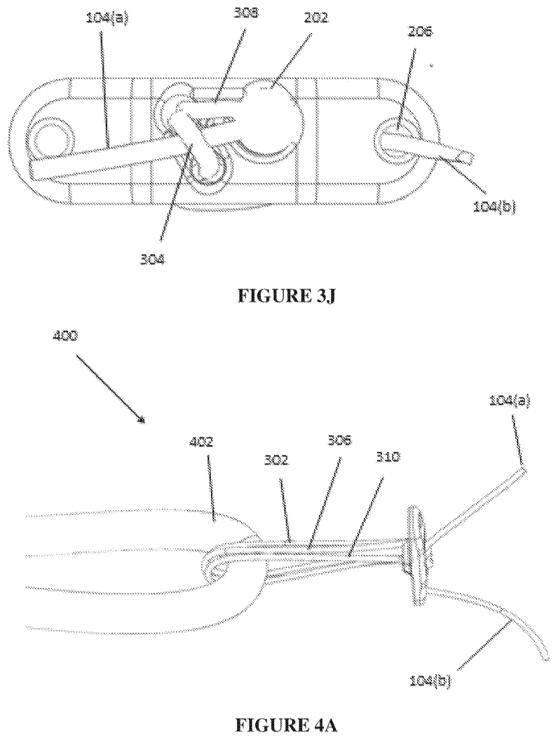

FIG. 3(a), 3(b), 3(c), 3(d), 3(e), 3(f), 3(g), 3(h), 3(i) and 3(j) illustrates the winding path of the flexible braided medical strand 104 with the fixation plate 102 through the plurality of holes 204, 206, twin apertures 210,212 and hole 216 with a step 218. The following steps are conducted to engage the flexible braided medical strand 104 to the fixation plate 102 as shown in FIG. 3(a), 3(b), 3(c), 3(d), 3(e), 3(f), 3(g), 3(h), 3(i) and 3(j).

As shown in FIG. 3(a), put the tension end 104a of the braided medical strand 104 into hole 216 from the upper side 102a of the fixation plate until the knot 202 gets arrested on step 218. Now put the tension end 104(a) of the flexible braided medical strand 104 from the lower side 102b of the fixation plate 102 into the aperture 210 to form a first adjustable fixation loop 302. (Ref. FIG. 3(b)). The tension end 104a of the flexible braided medical strand 104 is not limited to the aperture 210 and can be put in either of the apertures 210 or 212 from the lower side 102b of the fixation plate 102.

Now put the tension end 104a of the flexible braided medical strand 104 from the upper side 102a of the fixation plate 102 through the aperture 212 leaving a first small loop 304. (Explained later in the upcoming steps). (Ref. FIG. 3(c)).

Put the tension end 104a of the flexible braided medical strand 104 from the lower side 102b of the fixation plate 102 back through the hole 216 forming the second adjustable fixation loop 306 wherein both the tension end 104a and lead end 104b extendingng away from the upper side 102a of the fixation plate 102. (Ref. FIG. 3(d)).

Now, put the tension end 104a of the flexible braided medical strand 104 through the aperture 210 from the upper side 102a of the fixation plate 102 leaving a second small loop 308 as shown in FIG. 3(e). Now put the tension end 104a of the flexible braided medical strand 104 from the lower side 102b of the fixation plate 102 back through the hole 216 forming the third adjustable fixation loop 310. (Ref. FIG. 3(f)).

Now put the tension end 104a of the flexible braided medical strand 104 through the first small loop 304 to make a locking arrangement while the second small loop 308 gets fit into the channel 222 when the tension end 104(a) of braided medical strand 104 is pulled as shown in FIG. 3(g)

Now referring to FIG. 3(h)-3(j), pass the lead end 104b of the flexible braided medical strand 104 through the hole 216 via the guide way 224 at the lower side 102b of the fixation plate 102 and eventually pull out the lead side 104b through the second through hole 206 at the upper side 102a of the fixation plate 102.

FIG. 3(a)-3(j) are merely examples. A person skilled in the art will recognize many variations, alternatives, and modifications of the embodiment of the present disclosure.

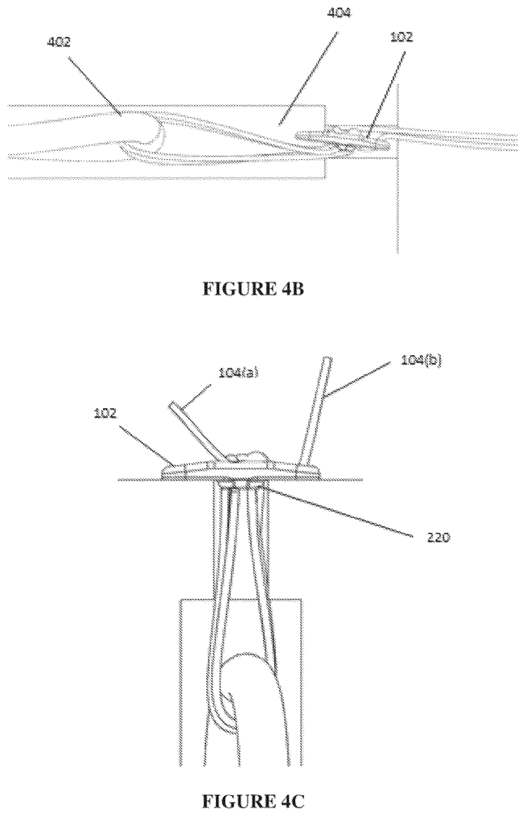

Referring to FIG. 4(a)-4(c), a graft 402 is attached to the first, second and third adjustable fixation loops 302,306 and 310 respectively of the fixation device 100 wherein the lead side 104b is pulled to bring the whole graft 402 into a bone tunnel 404. The curved edges of fixation plate 102 ensure the smooth pulling of the fixation plate 102 through tunnel 404 without damaging the bone (Ref. FIG. 4(b)). As shown in FIG. 4(c), once fixation plate 102 is pulled out of the bone tunnel, the fixation plate 102 sits comfortably at the end of the bone tunnel with respect to the stabilizing member 220.

The present subject matter facilitates the operation of fixation device 100 by activating a loop length adjustment mechanism which works substantially like principle of pulley to resize the graft supporting multiple adjustable loops by applying tension to the assembly in one direction and activating an automatic loop locking mechanism by applying tension to the assembly in the opposite direction.

For this during surgery, tension end 104b of the braided medical strand 104 is pulled which releases the lock arrangement and the arrested knot ball 202 in the hole upwards due to friction thereby making the length of third adjustable fixation loop 310 shorter. This shortening of the length of third adjustable fixation loop 310 transfers all the load of the graft on this loop. As a result of tension, the braided medical strand slides into the channel making the second adjustable fixation loop 306 move towards the upper side of fixation plate via the hole 216 making the length of second adjustable fixation loop 306 shorter and re-adjusting them to same lengths subsequently sharing the load of graft by third adjustable fixation loop 310 and second adjustable fixation loop 306. This results in the sliding of first adjustable fixation loop 302 through the bridge 214 towards the upper side 102(a) of the fixation plate 102 via the hole 216 making the length of first adjustable fixation loop 302 shorter and readjusting to the same length of other two fixation loops 306 and 310 respectively eventually sharing the equal load. This constant sliding of the adjustable fixation loops, particularly working on the principle of pulley mechanism results into the simultaneous and equal shortening of all three adjustable fixation loops. Once the graft 402 is securely suspended in the bone tunnel 404 and desired strength is achieved, the surgeon releases the tensioning side of braided medical strand, leading to arresting of knot ball 202 in the hole 216 and pinching the braided medical strand forming a lock arrangement properly by arresting the adjustable loop into the bridge 214. The extra length above the upper side of the fixation plate 102 of both the adjustable fixation loops 304 and 306 can be cutoff and removed.

It is observed that the use of multiple sutures or threads for the surgery has been eliminated.

It is further observed that along with the elimination of the multiple sutures, the single braided medical strand along with the fixation plate has provided the much-needed strength to the graft which avoids any further post-surgery problems as well as minimizing slip/creep of the braided medical strand within the loop. Along with these advantages, there is a cost reduction, as well as elimination of time-consuming surgery as described in the present subject matter.

The present subject matter has both technical as well as economic significance with respect to the conventional devices for fixation or the like.

While a particular embodiment of the present subject matter has been illustrated and described, modifications thereof will readily occur to those skilled in the art. It is understood that the various embodiment, details and constructions of the fixation device and their features described above and illustrated in the attached Figures may be interchanged among the various embodiment while remaining within the scope of the present subject matter. Additionally, it is understood that various modifications could be made to any of the fixation device and/or elements described herein above while remaining within the scope of the present subject matter.

1. A device for tissue repairs and reconstruction of anterior cruciate ligament (ACL) and posterior cruciate ligament (PCL), the device comprising:
a fixation plate having an upper side and a lower side, the upper side comprising a plurality of circular through holes, an elliptical groove with twin apertures connected by a bridge, and a hole with a step extending from the upper side to the lower side; and
a strand, the strand comprising a knot, wherein the knot divides the strand into a tension end and a lead end, wherein the tension end is passed through the hole in the fixation plate from the lower side to the upper side and through one of the twin apertures from the upper side to the lower side, forming a first adjustable fixation loop, wherein the tension end of the strand further passed through the aperture from the lower side to the upper side, forming a first small loop, wherein the tension end of the strand is passed through the hole from the lower side to the upper side, forming a second adjustable fixation loop with the tension end and lead end extending away from the upper side; wherein the tension end of the strand is further passed through the aperture from the upper side to the lower side, forming a second small loop; wherein the tension end of the strand is passed through the hole from the lower side to the upper side, forming a third adjustable fixation loop; and wherein the tension end of the strand is passed through the first small loop to lock the adjustable loops, and the lead end of the strand is pulled through the hole and a guide way at the lower side, then pulled out through a second through hole at the upper side.

2. The device of claim 1, wherein the device comprises a graft connected to the first, second, and third adjustable fixation loops, wherein tension applied to the tension end of the strand shortens the adjustable loops, achieving graft fixation and minimizing slip/creep of the strand within the loops

3. The device of claim 1, wherein the fixation plate has curved edges to facilitate smooth pulling of the fixation device through a bone tunnel during surgery.

4. The device of claim 1, wherein the circular through holes is disposed at corners of the fixation plate.

5. The device of claim 1, wherein the bridge connecting the twin apertures forms a twisted path for the strand.

6. The device of claim 1, wherein the step within the bole is configured to arrest the knot in the strand, preventing its slippage during tensioning.

7. The device of claim 1, wherein the tension end of the strand is slidably adjustable within the twin apertures to achieve desired tension in the graft.

8. The device of claim 1, wherein the tension end of the strand is pulled to shorten the adjustable loops, distributing the load on the graft, and achieving desired fixation.

9. The device of claim 1, wherein the fixation plate is made from a material selected from the group consisting of plastic, titanium alloy, and steel, and wherein the flexible single strand is made of Ultra High Molecular Weight polyethylene (UHMWPE) or a biocompatible material.

10. The device of claim 1, wherein the strand is flexible, single braided, medicated and shaped in a circular cross-section.

11. A method for attaching a graft comprising the steps of:
providing a device, the device comprising a fixation plate having an upper side and a lower side, the upper side comprising a plurality of circular through holes, an elliptical groove with twin apertures connected by a bridge, and a hole with a step extending from the upper side to the lower side, and a flexible single strand having a knot dividing it into a tension end and a lead end;
passing the tension end of the strand through the hole in the fixation plate from the lower side to the upper side, then through one of the twin apertures from the upper side to the lower side, forming a first adjustable fixation loop;
passing the tension end of the strand through the aperture from the lower side to the upper side, forming a first small loop;
passing the tension end of the strand through the hole from the lower side to the upper side, forming a second adjustable fixation loop with the tension end and lead end extending away from the upper side;
passing the tension end of the strand through the aperture from the upper side to the lower side, forming a second small loop;
passing the tension end of the strand through the hole from the lower side to the upper side, forming a third adjustable fixation loop;
passing the tension end of the strand through the first small loop to lock the adjustable loops, and pulling the lead end of the strand through the hole and a guide way at the lower side, then pulling the lead end out through a second through hole at the upper side;
attaching a graft to the first, second, and third adjustable fixation loops of the fixation device; and
applying tension to the tension end of the strand, shortening the adjustable loops, and transferring load onto the graft, thereby achieving graft fixation, and minimizing slip/creep of the strand within the loops.
